(19) United States
(12) Patent Application Publication
(10) Pub. No.: US 2024/0140795 A1
(43) Pub. Date: May 2, 2024

(54) Manufacturing Method and Manufacturing Apparatus of Syngas, and Manufacturing Method of Liquid Hydrocarbon Using the Same

(71) Applicant: SK Innovation Co, Ltd., Seoul (KR)

(72) Inventors: Hee Jung Jeon, Daejeon (KR); Ok Youn Kim, Daejeon (KR); Dong Min Yun, Daejeon (KR)

(73) Assignee: SK Innovation Co., Ltd., Seoul (KR)

(21) Appl. No.: 18/373,533

(22) Filed: Sep. 27, 2023

(30) Foreign Application Priority Data
Oct. 31, 2022 (KR) 10-2022-0142373

(51) Int. Cl. C01B 3/44; B01J 8/00; B01J 8/18; B01J 8/26; C01B 3/52; C01B 3/56; C01B 32/40; C07C 1/04; C10J 1/26

(52) U.S. Cl. CPC C01B 3/44; B01J 8/0055; B01J 8/1818; B01J 8/26; C01B 3/52; C01B 3/56; C01B 32/40; C07C 1/043; C10J 1/26; C01B 2203/0244; C01B 2203/0415; C01B 2203/042; C01B 2203/0475; C01B 2203/1058; C01B 2203/1082; C01B 2203/1241; C10J 2300/0946

(57) ABSTRACT

Provided is a method of manufacturing syngas including (S1) heat-treating organic wastes under a catalyst in a first reactor to produce a first mixed gas; (S2) separating the catalyst and carbon dioxide (CO₂) from the first mixed gas, and recovering a mixed gas from which the catalyst and the carbon dioxide (CO₂) have been removed; (S3) converting the carbon dioxide (CO₂) separated in (S2) into carbon monoxide (CO) by a reverse Boudouard reaction in a second reactor; and (S4) mixing the mixed gas recovered in (S2) and the carbon monoxide (CO) converted in (S3) to produce syngas.

### CROSS-REFERENCE TO RELATED APPLICATION

This application claims priority to Korean Patent Application No. KR 10-2022-0142373, filed Oct. 31, 2022, the disclosure of which is hereby incorporated by reference in its entirety.

### BACKGROUND OF THE INVENTION

### Field of the Invention

The present disclosure relates to a method and an apparatus for manufacturing syngas, and in some embodiments, to a method and an apparatus for manufacturing syngas which may increase a manufacturing yield of syngas and minimize carbon dioxide emission.

### Description of Related Art

Organic wastes may seriously damage the environment by decay when landfilled, and should be discarded through a prescribed treatment process after collecting by their properties when discarded. However, since simple disposal of the organic wastes requires securing treatment facilities and consuming a large amount of manpower and is more wasteful than productive, methods and technologies for recycling organic wastes have been developed in recent years. Representatively, a gasification process technology in which syngas is produced using organic wastes and converted into a high value-added product for energization, may be mentioned.

The gasification process generally refers to a series of processes of reacting carbonaceous raw materials such as coal, organic wastes, and/or biomass under the supply of water vapor, oxygen, carbon dioxide, or a mixture thereof to convert the raw materials into syngas formed of hydrogen and carbon monoxide as the main components, in which the “syngas” or “synthesis gas” refers to mixed gas which is usually produced by a gasification reaction and contains hydrogen and carbon monoxide as the main component and may further include carbon dioxide and/or methane.

The gasification process technology has expanded to a technology of producing fuels and raw materials of various compounds, and for example, the syngas may be used as the raw material of a Fischer-Tropsch synthesis reaction to manufacture high value-added products such as light oil, heavy oil, diesel oil, jet oil, and/or lube oil. Besides, it is known that hydrogen in the syngas which is the main product of the gasification process is used to be applied to hydrogen power generation, ammonia manufacture, an oil refining process, and/or the like, and methanol manufactured from the syngas may be used to obtain high value-added chemicals such as acetic acid, olefin, dimethylether, aldehyde, fuel, and/or an additive.

Recently, as a process for manufacturing syngas, a gasification process using a catalyst has been carried out, but due to the formation of coke and the like in the gasification process, the catalyst is inactivated which causes process trouble in continuous operation. In addition, for securing economic feasibility, relatively expensive catalysts need to be recovered, but in order to recover catalyst discharged in the state in which coke is agglomerated, a plurality of subsequent processes (such as hot-water extraction and lime digestion) should be carried out, and thus, process efficiency is significantly deteriorated.

In the gasification reaction of organic wastes, an additional process such as further supplying separate hydrogen may be performed for improving a manufacturing yield, but process efficiency is significantly low, and a separate water-gas shift reaction may be performed, but carbon dioxide as well as hydrogen is produced in a water-gas shift process to cause environmental pollution.

Since the conventionally performed gasification process of organic wastes has a significantly low manufacturing yield of syngas (H₂, CO) which may be converted into a high value-added product and has poor productivity, commercialization using the process is limited. Further, in terms of environmental protection, it is preferred to suppress CO₂ emission, but since the gasification reaction product of organic wastes contains CO₂ in addition to H₂ and CO, carbon dioxide emission is higher than that in landfill or pyrolysis treatment, and thus, the gasification process has a serious problem of rather causing more environmental pollution.

Thus, a method and an apparatus for manufacturing syngas which increase a manufacturing yield of syngas which may be converted into a high value-added product and minimize carbon dioxide formation in the gasification process of organic wastes are needed.

### SUMMARY OF THE INVENTION

In some embodiments, the present disclosure is directed to providing a method and an apparatus for manufacturing syngas which have a significantly improved manufacturing yield of syngas from organic wastes.

In some embodiments, the present disclosure is directed to providing a method and an apparatus for manufacturing syngas which may minimize carbon dioxide formation.

In addition, the technical objects to be achieved in the present disclosure are not limited to the above-mentioned technical objects, and another objects which are not mentioned may be clearly understood by those skilled in the art to which the present invention pertains from the following description.

In some embodiments, a method for manufacturing syngas comprises: (S1) heat-treating organic wastes under a catalyst in a first reactor to produce a first mixed gas; (S2) separating the catalyst and carbon dioxide (CO₂) from the first mixed gas, and recovering a second mixed gas from which the catalyst and the carbon dioxide (CO₂) have been removed; (S3) converting the carbon dioxide (CO₂) separated in (S2) into carbon monoxide (CO) by a reverse Boudouard reaction in a second reactor; and (S4) mixing the second mixed gas recovered in (S2) and the carbon monoxide (CO) converted in (S3) to produce syngas.

In some embodiments, in (S1), when the first mixed gas is produced, a methane reforming reaction in which methane is converted into carbon and hydrogen may occur simultaneously.

In some embodiments, the first mixed gas produced in (S1) may comprise hydrogen (H₂), carbon monoxide (CO), and/or carbon dioxide (CO₂).

In some embodiments, (S3) may further comprise (S3-1) introducing the catalyst separated in (S2) to the second reactor and regenerating the catalyst through a reverse Boudouard reaction; and (S3-2) recirculating and resupplying the regenerated catalyst to (S1).

In some embodiments, in (S1), the catalyst may be a composite catalyst in which at least one active metal selected from nickel, iron, or vanadium is supported on a support.

In some embodiments, in (S2), the carbon dioxide (CO₂) may be adsorbed on an adsorbent comprising at least one selected from calcium oxide (CaO), calcium hydroxide (Ca(OH)₂), dolomite, limestone, or trona and separated.

In some embodiments, the adsorption of carbon dioxide (CO₂) may be performed at a temperature of 500° C. or higher and lower than 800° C. and/or a pressure of 50 to 200 kPa.

In some embodiments, (S1) may be performed at a temperature of 600 to 900° C. and/or a pressure of 50 to 200 kPa.

In some embodiments, the reverse Boudouard reaction of (S3) may be performed at a temperature of 800 to 1000° C. and/or a pressure of 50 to 200 kPa.

In some embodiments, the syngas produced in (S4) may comprise hydrogen (H₂) and carbon monoxide (CO) and a molar ratio between the hydrogen (H₂) and the carbon monoxide (CO) may satisfy 1.8 to 2.2.

In some embodiments, the molar ratio between the hydrogen (H₂) and the carbon monoxide (CO) may be adjusted by controlling the reverse Boudouard reaction depending on a flow rate of the carbon monoxide (CO) converted in (S3).

In some embodiments, the molar ratio between the hydrogen (H₂) and the carbon monoxide (CO) may be adjusted by supplying separate carbon dioxide to (S3) depending on a flow rate of the carbon monoxide (CO) converted in (S3).

In some embodiments, the organic wastes in (S1) may be at least one selected from waste plastic, solid wastes, biomass, waste oil, or waste tires.

In some embodiments, before (S2), the first mixed gas produced in (S1) may be purified.

In some embodiments, an apparatus for manufacturing syngas comprises: a first reactor where organic wastes are introduced and a methane reforming reaction and a gasification reaction are simultaneously performed under a catalyst to form a product; a carbon dioxide separation unit where the product is introduced from the first reactor and carbon dioxide is separated; a second reactor where the carbon dioxide separated from the carbon dioxide separation unit is introduced and a reverse Boudouard reaction is performed; and a syngas production unit where a product from which the carbon dioxide has been removed in the carbon dioxide separation unit and carbon monoxide converted in the second reactor are mixed to produce syngas.

In some embodiments, the first reactor may comprise a fluidized bed reactor.

In some embodiments, the second reactor may comprise a fluidized bed reactor.

In some embodiments, the apparatus for manufacturing syngas may further comprise a purification unit between the first reactor and a carbon dioxide storage unit.

In some embodiments, the apparatus for manufacturing syngas may further comprise a cyclone which separates the catalyst from the product of the first reactor between the first reactor and the carbon dioxide separation unit; a supply line which supplies the catalyst separated from the cyclone to the second reactor; and a recirculation line which resupplies the regenerated catalyst from the second reactor to the first reactor.

In some embodiments, a method for manufacturing a liquid hydrocarbon comprises: supplying syngas to a third reactor; and performing a Fischer-Tropsch synthesis reaction in the third reactor, wherein the syngas is the syngas manufactured by a method disclosed herein.

In some embodiments, the liquid hydrocarbon may comprise naphtha having a boiling point of 150° C. or lower, Kero having a boiling point of 150 to 265° C., LGO having a boiling point of 265 to 340° C., and/or VGO having a boiling point of 340° C. or higher.

Other features and aspects will be apparent from the following detailed description, the drawings, and the claims.

### BRIEF DESCRIPTION OF THE DRAWINGS

FIG. 1 illustrates a block diagram of a method for manufacturing syngas according to some embodiments of the present disclosure comprising a first reactor, a carbon dioxide separation unit, a second reactor, and a syngas production unit.

FIG. 2 illustrates a block diagram of a method for manufacturing syngas according to some embodiments of the present disclosure comprising a first reactor, a purification unit, a carbon dioxide separation unit, a second reactor, and a syngas production unit.

FIG. 3 illustrates a block diagram of a method for manufacturing syngas according to some embodiments of the present disclosure comprising a first reactor, a cyclone, a purification unit, a carbon dioxide separation unit, a second reactor, and a syngas production unit.

FIG. 4 illustrates a block diagram of a method for manufacturing a liquid hydrocarbon according to some embodiments of the present disclosure comprising a first reactor, a carbon dioxide separation unit, a second reactor, a syngas production unit, and a third reactor.

### DETAILED DESCRIPTION OF MAIN ELEMENTS

1: feed, 100: first reactor, 200: carbon dioxide separation unit, 300: second reactor, 400: syngas production unit, 500: purification unit, 600: cyclone, 700: third reactor

### DESCRIPTION OF THE INVENTION

The singular form used in the present specification and claims appended thereto may be intended to include a plural form also, unless otherwise indicated in the context. As used herein, the singular form of “a”, “an”, and “the” include plural referents unless the context clearly states otherwise.

The numerical range used in the present specification comprises all values within the range including the lower limit and the upper limit, increments logically derived in a form and spanning in a defined range, all double limited values, and all possible combinations of the upper limit and the lower limit in the numerical range defined in different forms. Unless otherwise defined in the present specification, values which may be outside a numerical range due to experimental error or rounding off of a value are also included in the defined numerical range.

Also, it should be understood that any numerical range recited herein is intended to include all sub-ranges subsumed therein. For example, a range of “1 to 10” is intended to include any and all sub-ranges between and including the recited minimum value of 1 and the recited maximum value of 10, that is, all subranges beginning with a minimum value equal to or greater than 1 and ending with a maximum value equal to or less than 10, and all subranges in between, e.g., 1 to 6.3, or 5.5 to 10, or 2.7 to 6.1.

For the purposes of this disclosure, unless otherwise indicated, all numbers expressing quantities of ingredients, reaction conditions, dimensions, physical characteristics, and so forth used in the disclosure are to be understood as being modified in all instances by the term “about.” Hereinafter, unless otherwise particularly defined in the present disclosure, “about” may be considered as a value within 30%, 25%, 20%, 15%, 10%, 5%, 3%, 2%, 1%, 0.5%, 0.1%, 0.05% or 0.01% of a stated value. Unless indicated to the contrary, the numerical parameters set forth in this disclosure are approximations that can vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

In the present disclosure, the terms such as “comprise”, “include”, “contain” or “have” mean that there is a characteristic or a constitutional element described in the specification, and as long as it is not particularly limited, a possibility of adding one or more other characteristics or constitutional elements is not excluded in advance.

The unit of % used in the present specification without particular mention refers to % by weight, unless otherwise defined.

The unit of ppm used without particular mention in the present specification refers to ppm by mass, unless otherwise defined.

A boiling point used without particular mention in the present specification refers to a boiling point at 25° C. under 1 atm.

A density used without particular mention in the present specification refers to a density at 25° C. under 1 atm.

“Gasification” which is used without particular mention in the present specification refers to a thermal-chemical conversion process through a chemical structure change of a carbonaceous material in the presence of a gasifier (air, oxygen, steam, carbon dioxide, or a mixture thereof) in a broad sense, and refers to a process of converting the carbonaceous material mainly into syngas in a narrower sense.

As a conventional process for manufacturing syngas, a gasification process using a catalyst is carried out, but due to the formation of coke and the like in the gasification process, the catalyst is inactivated which causes process trouble in continuous operation. In addition, for securing economic feasibility, relatively expensive catalysts need to be recovered, but in order to recover catalyst discharged in the state in which coke is agglomerated, a plurality of subsequent processes (such as hot-water extraction and lime digestion) should be carried out, and thus, process efficiency is significantly deteriorated.

In the case of syngas produced by the gasification reaction, an additional process such as further supplying separate hydrogen may be performed for improving a manufacturing yield, but process efficiency is significantly low, and an additional water-gas shift reaction may be performed, but carbon dioxide as well as hydrogen is produced in the water-gas shift reaction to cause environmental pollution.

Since the conventionally performed gasification process of organic wastes has a significantly low manufacturing yield of syngas which may be converted into a high value-added product of 30% or less and has poor productivity, commercialization using the process is limited. Further, in terms of environmental protection, it is preferred to suppress CO₂ emission, but since the gasification reaction product of organic wastes contains CO₂ in addition to H₂ and CO, carbon dioxide emission is higher than that in landfill or pyrolysis treatment, and thus, the gasification process has a serious problem of rather causing more environmental pollution.

Thus, the present disclosure provides a method of manufacturing syngas comprising: (S1) heat-treating organic wastes under a catalyst in a first reactor to produce a first mixed gas; (S2) separating the catalyst and carbon dioxide (CO₂) from the first mixed gas, and recovering a second mixed gas from which the catalyst and the carbon dioxide (CO₂) have been removed; (S3) converting the carbon dioxide (CO₂) separated in (S2) into carbon monoxide (CO) by a reverse Boudouard reaction in a second reactor; and (S4) mixing the second mixed gas recovered in (S2) and the carbon monoxide (CO) converted in (S3) to produce syngas. Formation of carbon dioxide is minimized by the manufacturing method as compared with the conventional method, thereby preventing environmental pollution and significantly improving a manufacturing yield of syngas from organic wastes.

In (S1), the organic wastes are heat treated under a catalyst in a first reactor to produce a first mixed gas, and usually, at least one reaction selected from the following Reaction Formulae 1 to 4 is involved to produce the first mixed gas.

C+H₂O→H₂+CO (water gasification reaction of carbon) [Reaction Formula 1]

C+CO₂→2CO (carbon dioxide gasification reaction of carbon) [Reaction Formula 2]

CO+3H₂→CH₄+H₂O (methanation reaction) [Reaction Formula 3]

C+O₂→CO₂ (oxidation reaction of carbon). [Reaction Formula 4]

In some embodiments, in (S1), when the first mixed gas is produced, a methane reforming reaction in which methane is converted into carbon and hydrogen may occur simultaneously. In some embodiments, the first mixed gas produced in (S1) may comprise hydrogen (H₂), carbon monoxide (CO), and/or carbon dioxide (CO₂). In some embodiments, the product of (S1) may comprise H₂, CO, CH₄, and/or CO₂ comprising moisture, and when the first mixed gas is produced, the methane reforming reaction occurs simultaneously, and thus, production yields of H₂ and CO which are the main components of syngas which may be converted into high value-added products may be significantly improved. The methane reforming reaction may involve at least one selected from the following Reaction Formulae 5 to 7:

CH₄→C+2H₂ (methane reforming reaction) [Reaction Formula 5]

CH₄+H₂O→CO+3H₂ (steam modification reaction of methane) [Reaction Formula 6]

CH₄+CO₂→2CO+2H₂ (carbon dioxide reforming reaction of methane). [Reaction Formula 7]

It is favorable that (S1) is performed under a water vapor atmosphere by supplying the water vapor atmosphere to the first reactor. For improving reaction efficiency, a small amount of carbon dioxide may be also optionally supplied in addition to the water vapor, and the water vapor amount in the first reactor may be adjusted by adjusting partial pressure using oxygen and, if required, inert gas such as nitrogen and argon. As described later, (S1) may be performed at a temperature of 600 to 900° C. and/or a pressure of 50 to 200 kPa. Under these conditions, gasification efficiency and methane reformation efficiency may be excellent. In some embodiments, the temperature may be 700 to 900° C. and/or the pressure may be 100 to 200 kPa, or the temperature may be 850 to 900° C. and/or the pressure may be 150 to 200 kPa.

Since the methane reforming reaction of Reaction Formulae 5 to 7 occurs simultaneously with the gasification reaction of Reaction Formulae 1 to 4 in (S1), it is very favorable in terms of economic efficiency and/or energy efficiency while solving the problem of a significantly low manufacturing yield of syngas during the conventional gasification process of organic wastes. Since the syngas is manufactured by a series of processes comprising (S1) to (S4) described later, carbon dioxide emission is minimized to prevent environmental pollution and/or the manufacturing yield of syngas may be maximized.

The first reactor may comprise a fluidized bed reactor. It is preferred to use the fluidized bed reactor in terms of gasification efficiency and/or a catalyst regeneration process described later. In the fluidized bed reactor, a solid bed (bed material) is fluidized and mixed in a suspended state due to reaction gas having an upward flow to cause a gasification reaction. In some embodiments, the first reactor may comprise a fluidized bed reactor, for example in which the fluidized bed reactor may be a riser.

Through the process of (S2), in order to recover syngas from the product of (S1), the catalyst and the carbon dioxide (CO₂) are separated, and the second mixed gas from which the catalyst and the carbon dioxide (CO₂) have been removed may be recovered. In the separation of the catalyst and carbon dioxide (CO₂), it is preferred to separate the catalyst first and then separate carbon dioxide (CO₂), but the present disclosure is not necessarily limited thereto, and the catalyst and carbon dioxide (CO₂) may be separated at the same time. Preferably, as described later, it is preferred that a cyclone is provided between the first reactor and a carbon dioxide separation unit to separate the catalyst from the product of the first reactor (first mixed gas), and then the product from which the catalyst has been separated is introduced to the carbon dioxide separation unit to separate carbon dioxide and recover the second mixed gas.

Through the process of (S3), the carbon dioxide (CO₂) separated in (S2) may be converted into carbon monoxide (CO) by a reverse Boudouard reaction in the second reactor. The carbon dioxide produced by the heat treatment process of (S1) is not discharged as it is, but is converted into carbon monoxide, thereby preventing environmental pollution and also improving the manufacturing yield of syngas. The reverse Boudouard reaction may involve the reaction of the following Reaction Formula 8. In order to perform the reverse Boudouard reaction well, carbon such as activated carbon may be supplied, or as described later, in (S1), a catalyst inactivated by coke and/or the like is used to perform the reverse Boudouard reaction.

C+CO₂→2CO (reverse Boudouard reaction) [Reaction Formula 8]

The carbon monoxide which has been converted by the reverse Boudouard reaction of (S3) may be used in the production of syngas in (S4) described later. When unreacted carbon dioxide is present in the product of (S3), carbon dioxide is separated again from the product, and the reverse Boudouard reaction of (S3) may be repeated once or more on the separated carbon dioxide. Accordingly, carbon dioxide may be converted into carbon monoxide in a very high yield.

Through the process of (S4), the second mixed gas recovered in (S2) and the carbon monoxide (CO) converted in (S3) may be mixed to produce syngas. As described above, the syngas contains CO and H₂ as main components, and the syngas is used as a raw material in a Fischer-Tropsch reaction to manufacture high value-added products, in which the stoichiometrically required molar ratio of H₂:CO is preferably about 2:1, although this ratio is not required.

In the present disclosure, since syngas is manufactured by a series of processes comprising (S1) to (S4), the manufacturing yield of syngas is significantly increased as compared with the conventional gasification process of the organic wastes and/or carbon dioxide emission is minimized to prevent environmental pollution.

In some embodiments, (S3) may further comprise (S3-1) introducing the catalyst separated in (S2) to the second reactor and regenerating the catalyst through a reverse Boudouard reaction; and (S3-2) recirculating and resupplying the regenerated catalyst to (S1). As described above, the catalyst is inactivated by occurrence of coke and the like which are by-products in the conventional gasification process, so that process trouble occurs by the inactivated catalyst during continuous operation. In order to solve the problem, a certain amount of a new catalyst is continuously exchanged with a waste catalyst to maintain a catalytic activity at a certain level or higher, but process efficiency is significantly deteriorated in the exchange process. In the present disclosure, carbon dioxide is converted into carbon monoxide in the reverse Boudouard reaction, and simultaneously a waste catalyst in which coke and the like are agglomerated is treated with the carbon dioxide to regenerate the catalyst, which is recirculated to (S1), resupplied, and used, thereby converting carbon dioxide emitted to the outside into carbon monoxide to improve the manufacturing yield of syngas and also promoting the regeneration of the waste catalyst to significantly improve process efficiency. Herein, the second reactor may comprise the fluidized bed reactor, and for example, a regenerator in the fluidized bed reactor.

In some embodiments, in (S1), the catalyst may be a composite catalyst in which at least one active metal selected from nickel, iron, or vanadium is supported on a support. The support may be any support having durability to support active metal, and for example, may be selected from silicon, aluminum, zircon, sodium, manganese, titanium, oxides thereof, and/or the like. In some embodiments, in terms of gasification and methane reforming efficiency, the active metal may be nickel, and the support may be silicon dioxide.

In some embodiments, in (S2), the carbon dioxide (CO₂) may be adsorbed on an adsorbent comprising at least one selected from calcium oxide (CaO), calcium hydroxide (Ca(OH)₂), dolomite, limestone, or trona, and separated. Carbon dioxide may be effectively separated from the first mixed gas by the adsorbent. In some embodiments, the adsorbent may be calcium oxide.

The adsorption of carbon dioxide (CO₂) may be performed at a temperature of 500° C. or higher and lower than 800° C. and/or a pressure of 50 to 200 kPa. Under the conditions, carbon dioxide adsorption efficiency may be excellent. In some embodiments, the temperature may be 500 to 700° C. and/or the pressure may be 50 to 150 kPa, or the temperature may be 550 to 650° C. and/or the pressure may be 50 to 100 kPa.

The carbon dioxide adsorbed on the adsorbent is desorbed again and may be used as a reactant of the reverse Boudouard reaction of (S3). The desorption may be performed at a temperature of higher than 500 and 1000° C. or less and/or a pressure of 50 to 200 kPa. Under these conditions, carbon dioxide desorption efficiency may be excellent. In some embodiments, the temperature may be 700 to 950° C. and/or the pressure may be 50 to 150 kPa, or the temperature may be 850 to 950° C. and/or the pressure may be 50 to 100 kPa.

In some embodiments, (S1) may be performed at a temperature of 600 to 900° C. and/or a pressure of 50 to 200 kPa. Under these conditions, gasification efficiency and/or methane reformation efficiency may be excellent. In some embodiments, the temperature may be 700 to 900° C. and/or the pressure may be 100 to 200 kPa, or the temperature may be 850 to 900° C. and/or the pressure may be 150 to 200 kPa.

In some embodiments, the reverse Boudouard reaction of (S3) may be performed at a temperature of 800 to 1000° C. and/or a pressure of 50 to 200 kPa. Under the conditions, the conversion efficiency from carbon dioxide into carbon monoxide and the catalyst regeneration efficiency may be excellent. In some embodiments, the temperature may be 800 to 1000° C. and/or the pressure may be 50 to 150 kPa, or the temperature may be 950 to 1000° C. and/or the pressure may be 100 to 150 kPa.

In some embodiments, the syngas produced in (S4) may comprise hydrogen (H₂) and carbon monoxide (CO) and a molar ratio between the hydrogen (H₂) and the carbon monoxide (CO) may satisfy 1.8 to 2.2. As described above, the syngas is used as a raw material of the Fischer-Tropsch reaction and converted into high value-added products such as light oil, heavy oil, diesel oil, jet oil, and/or lube oil, and when a stoichiometrically required molar ratio of H₂:CO satisfies 1.8 to 2.2, conversion efficiency may be excellent. Preferably, the molar ratio of H₂:CO may be 1.9 to 2.1, or 1.95 to 2.05.

In some embodiments, the molar ratio between the hydrogen (H₂) and the carbon monoxide (CO) may be adjusted by controlling the reverse Boudouard reaction depending on a flow rate of the carbon monoxide (CO) converted in (S3). A flow rate of the carbon monoxide converted in (S3) is measured in real time and the reverse Boudouard reaction is controlled so that the molar ratio of H₂:CO is satisfied. The reaction conditions and the reaction rate of the reverse Boudouard reaction in the second reactor are controlled, thereby satisfying the H₂:CO molar ratio to improve process efficiency. In addition, when the flow rates of H₂ and CO in the second mixed gas recovered in (S2) are measured together, process efficiency may be more improved.

In some embodiments, the molar ratio between the hydrogen (H₂) and the carbon monoxide (CO) may be adjusted by controlling the reverse Boudouard reaction by supplying separate carbon dioxide to (S3) depending on a flow rate of the carbon monoxide (CO) converted in (S3). A flow rate of the carbon monoxide converted in (S3) is measured in real time so that the molar ratio of H₂:CO is satisfied and separate carbon dioxide may be supplied and adjusted. Specifically, when CO is significantly small in the molar ratio of H₂:CO, carbon dioxide is separately supplied to (S3), thereby improving the reverse Boudouard reaction activity to satisfy the molar ratio of H₂:CO. The supply of carbon dioxide may be performed by a separate carbon dioxide storage unit.

In some embodiments, the organic wastes in (S1) may be at least one selected from waste plastic, solid wastes, biomass, waste oil, and/or waste tires. Without being necessarily limited thereto, any carbon-containing material such as biomass and/or coal may be used without limitation as the raw material of a gasification reaction, but considering the problem to be solved in the present disclosure, it may be appropriate to use the organic wastes.

In some embodiments, before (S2), the first mixed gas produced in (S1) may be further purified. The first mixed gas produced in (S1) may further comprise aqueous impurity gas components such as H₂S, HCl, HOCl, and/or NH₃, in addition to H₂, carbon dioxide (CO), and carbon dioxide (CO₂). Thus, impurity gas components are removed by the process of purifying the first mixed gas, by which a manufacturing yield of syngas may be further improved. In some embodiments, before (S2), when H₂ is relatively insufficiently produced in the gasification process depending on the kind of organic wastes, a water gas shift (WGS) reaction is performed to replenish H₂, thereby improving the manufacturing yield of syngas. The WGS reaction may be performed in the conditions of 20 to 70 bar and/or 100 to 250° C. under a catalyst. The catalyst may be used without limitation as long as it is a catalyst having WGS reaction activity, and preferably, may be a Cu—Zn mixed catalyst. Herein, since carbon dioxide produced by the water gas shift reaction is converted into carbon monoxide by the reverse Boudouard reaction, the environmental pollution problem caused by performing the conventional water gas shift reaction may be solved and simultaneously the effect of improving a manufacturing yield of syngas may be promoted.

In some embodiments, the present disclosure provides an apparatus for manufacturing syngas comprising: a first reactor 100 where organic wastes are introduced and a methane reforming reaction and a gasification reaction are simultaneously performed under a catalyst to produce a product; a carbon dioxide separation unit 200 where the product is introduced from the first reactor 100 and carbon dioxide is separated; a second reactor 300 where the carbon dioxide separated from the carbon dioxide separation unit 200 is introduced and a reverse Boudouard reaction is performed; and a syngas production unit 400 where a product from which the carbon dioxide has been removed in the carbon dioxide separation unit 200 and carbon monoxide converted in the second reactor 300 are mixed to produce syngas. The syngas may be manufactured in a high yield from the organic wastes by the apparatus, and carbon dioxide emission is minimized to prevent environmental pollution.

In some embodiments, the first reactor 100 may comprise a fluidized bed.

In some embodiments, the second reactor 300 may comprise a fluidized bed.

In the fluidized bed reactor, a solid bed (bed material) is fluidized and mixed in a suspended state due to reaction gas having an upward flow to cause a gasification reaction. In some embodiments, the first reactor 100 may be a riser in the fluidized bed reactor. Organic wastes are supplied into the first reactor 100, and may be pyrolyzed under a catalyst provided in the reactor or pyrolyzed under a catalyst which is supplied through a resupply line connected from the second reactor 300 to the first reactor 100 and regenerated. Herein, the second reactor 300 may be a regenerator in the fluidized bed reactor.

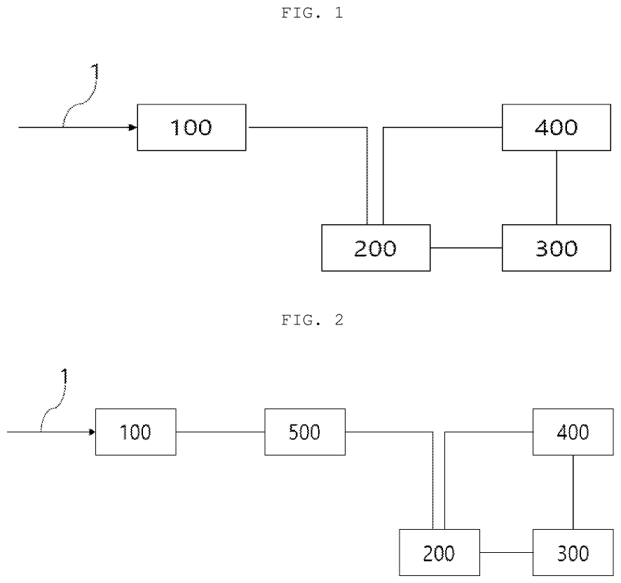

As shown in FIG. 1, an organic waste 1 is introduced into the first reactor and heat-treated under a catalyst to perform the gasification reaction and the methane reforming reaction simultaneously. The product (first mixed gas) produced in the first reactor 100 may comprise hydrogen (H₂), carbon monoxide (CO), and carbon dioxide (CO₂). The product is introduced from the first reactor 100 to the carbon dioxide separation unit 200 to separate carbon dioxide from the product.

The separated carbon dioxide may be introduced from the carbon dioxide separation unit 200 to the second reactor 300 and converted into carbon monoxide by the reverse Boudouard reaction.

The product from which carbon dioxide has been removed in the carbon dioxide separation unit 200 and the carbon monoxide converted in the second reactor 300 may be mixed in the syngas production unit 400 to produce the syngas. Herein, when unreacted carbon dioxide remains in the product of the second reactor 300, the product is introduced again to the carbon dioxide separation unit 200 to separate carbon dioxide from the product, and the separated carbon dioxide may be introduced again to the second reactor 300 to perform the reverse Boudouard reaction again in the second reactor 300. The process may be repeated once or more, thereby converting unreacted carbon dioxide into carbon monoxide to increase a carbon monoxide conversion rate.

In some embodiments, a purification unit 500 may be further comprised between the fluidized bed reactor and the carbon dioxide storage unit, as shown in FIG. 2. In the purification unit 500, the purification may be performed by a dust collection filter for high temperature/high pressure or a wet scrubber, and aqueous impurity gas components such as H₂S, HCl, HOCl, and/or NH₃ are removed to further improve the manufacturing yield of syngas.

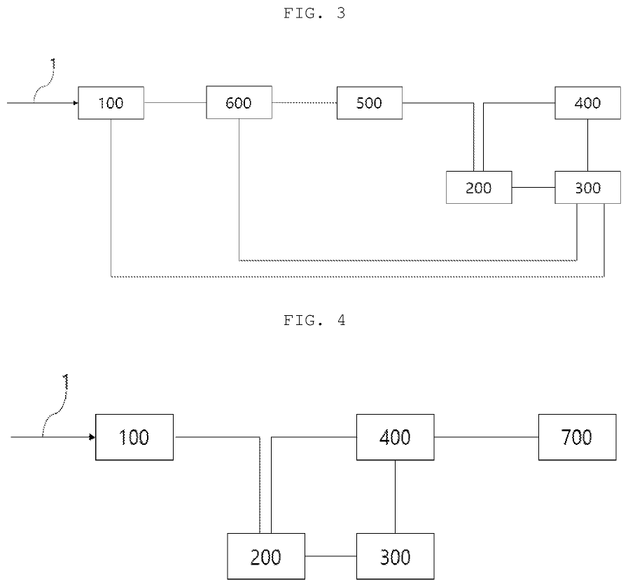

In some embodiments, the apparatus for manufacturing syngas may further comprise a cyclone 600 which separates the catalyst from the product of the first reactor 100; a supply line which supplies the catalyst separated from the cyclone 600 to the second reactor 300; and a recirculation line which resupplies the regenerated catalyst from the second reactor 300 to the first reactor 100, as shown in FIG. 3. The catalyst separated from the cyclone 600 is introduced to the second reactor 300, in which the catalyst may comprise a coke component produced in the pyrolysis process of the organic wastes. In some embodiments, carbon dioxide may be introduced from the carbon dioxide separation unit 200 to the second reactor 300. In some embodiments, the separate carbon dioxide storage unit may be provided to introduce separate carbon dioxide. The catalyst is treated with carbon dioxide at a high temperature of 800° C. or higher, thereby removing impurities such as coke comprised in the catalyst and regenerating the catalyst. The regenerated catalyst may be reintroduced to the first reactor 100 through the recirculation line.

In some embodiments, the present disclosure provides a method for manufacturing a liquid hydrocarbon comprising: supplying syngas to a third reactor 700; and performing a Fischer-Tropsch synthesis reaction in the third reactor 700, wherein the syngas is the syngas produced by any of the methods disclosed herein. As shown in FIG. 4, in the third reactor 700, the syngas may be supplied from the syngas production unit 400, and the syngas produced through (S1) to (S4) may be used as a raw material of the Fischer Tropsch reaction represented by the following Reaction Formula 9, thereby manufacturing a liquid hydrocarbon.

nCO+2nH₂→CₙH₂ₙ+nH₂O [Reaction Formula 9]

The third reactor 700 may be a Fischer-Tropsch synthesis reactor, in which the third reactor 700 may comprise a liquid product separation and a heavy end recovery unit. In some embodiments, a fixed bed reactor or a micro channel reactor may be applied to the third reactor 700, but the present disclosure is not necessarily limited thereto. In order to improve a manufacturing yield of the liquid hydrocarbon, it is preferred that a stoichiometric molar ratio of H₂:CO satisfies 1.8 to 2.2. In some embodiments, the molar ratio of H₂:CO may be 1.9 to 2.1, or 1.95 to 2.05. The manufactured liquid hydrocarbon may comprise naphtha having a boiling point of 150° C. or lower, Kero having a boiling point of 150 to 265° C., LGO having a boiling point of 265 to 340° C., and/or VGO having a boiling point of 340° C. or higher. The syngas produced in a conventional gasification process of organic wastes has a low amount of syngas (H₂, CO) which may be converted into a product (liquid hydrocarbon) and a lower molar mass of H₂ compared to CO in H₂:CO, and thus, a recovery rate of the expected product is significantly deteriorated. The syngas produced by the steps of (S1) to (S4) of the present disclosure is used as the raw material of the Fischer-Tropsch synthesis reaction, thereby improving the recovery rate of the liquid hydrocarbon product as compared with the conventional gasification process of organic wastes.

For the matters which are not described in the apparatus of manufacturing syngas of the present disclosure, the description herein of the method of manufacturing syngas may be referred.

Hereinafter, the present disclosure will be described in detail by the examples, however, the examples are for describing the present disclosure more detail, and the scope of rights is not limited to the following examples.

### Example 1

A syngas manufacturing apparatus comprising a first reactor, a purification unit, a carbon dioxide separation unit, a second reactor, and a syngas production unit was operated for 120 minutes to manufacture syngas. Specifically, 1000 g of municipal solid wastes was added to the first reactor (fluidized bed reactor) equipped with a Ni/SiO₂ catalyst, and then heat-treated at a temperature of 800° C. and a pressure of 100 kPa under water vapor conditions. The product of the first reactor (first mixed gas) was separated from the catalyst, and the product separated from the catalyst was added to the purification unit (wet scrubber) and treated at 70° C. and 100 kPa to remove impurities. Thereafter, the product was added to a carbon dioxide separation unit equipped with CaO to separate carbon dioxide. Specifically, the carbon dioxide was adsorbed to CaO at 650° C. and 100 kPa and separated, and the product from which the carbon dioxide has been removed (second mixed gas) was separately recovered. The CaO was treated again under the conditions of 850° C. and 100 kPa to desorb the carbon dioxide. The desorbed carbon dioxide was added to the second reactor (regenerator) and a reverse Boudouard reaction was carried out under conditions of 900° C. and 100 kPa to convert the carbon dioxide into carbon monoxide.

The second mixed gas from which the carbon dioxide has been removed and the carbon monoxide converted in the second reactor were introduced to a syngas production unit and mixed at 200° C. to manufacture syngas. In this process, a flow rate of the carbon monoxide converted in the second reactor was measured in real time, the activity of the reverse Boudouard reaction was adjusted depending on the amount of the converted carbon monoxide, and synthesis was performed so that a molar ratio of H₂:CO was 2 when syngas was produced.

In addition, the separated catalyst was supplied to the second reactor (regenerator) through a separate supply line, treated with the reverse Boudouard reaction, regenerated, and then resupplied to the first reactor through a recirculation line connected to the first reactor.

### Example 2

Syngas was manufactured in the same manner as in Example 1, except that the reaction was performed at a temperature of 850° C. and pressure of 150 kPa in the first reactor and a temperature of 950° C. and a pressure of 150 kPa in the second reactor.

### Example 3

Syngas was manufactured in the same manner as in Example 1, except that when the syngas was manufactured, the reaction was performed without measuring the flow rate of the carbon monoxide converted in the second reactor in real time.

### Example 4

Syngas was manufactured in the same manner as in Example 1, except that the reaction was performed without the recirculation of the catalyst without performing the regeneration process of the separated catalyst.

### Comparative Example 1

Syngas was manufactured in the same manner as in Example 1, except that the heat treatment process of the first reactor was performed without the catalyst.

### Comparative Example 2

Syngas was manufactured in the same manner as in Example 1, except that the reaction was performed without performing the reverse Boudouard reaction using the manufacturing apparatus of syngas including no second reactor.

### Evaluation Example

Gas chromatography (GC) was performed on the syngas manufactured by operating manufacturing apparatus of syngas for 120 minutes to analyze the composition. Specifically, the syngas was quantified by GC to calculate selectivity for each gas, and the composition of each gas was analyzed by the total amount of gas confirmed from a flowmeter, thereby evaluating the gas manufacturing yield and a carbon dioxide reduction effect.

The evaluation results are shown in the following Table 1:

TABLE 1

| | | Example | Example | Example | Example | Comparative | Comparative |
|---|---|---|---|---|---|---|---|
| | | 1 | 2 | 3 | 4 | Example 1 | Example 2 |
| H₂ | Molar rate | 1006 | 1128 | 1001 | 930 | 725 | 925 |
| | (lbmol/hr) | | | | | | |
| | Composition | 45 | 48 | 45 | 44 | 36 | 43 |
| | (vol %) | | | | | | |
| CO | Molar rate | 503 | 574 | 478 | 459 | 412 | 210 |
| | (lbmol/hr) | | | | | | |
| | Composition | 23 | 24 | 22 | 21 | 21 | 10 |
| | (vol %) | | | | | | |
| CO₂ | Molar rate | 609 | 548 | 624 | 635 | 656 | 884 |
| | (lbmol/hr) | | | | | | |
| | Composition | 27 | 24 | 28 | 30 | 32 | 41 |
| | (vol %) | | | | | | |
| Others | Molar rate | 110 | 95 | 107 | 112 | 197 | 135 |
| | (lbmol/hr) | | | | | | |
| | Composition | 5 | 4 | 5 | 5 | 11 | 6 |
| | (vol %) | | | | | | |

Referring to the results of analyzing the syngas compositions of Table 1, in the manufacturing process of syngas, Examples 1 to 4 had much better production amounts of H₂ and CO, and had reduced production amounts of CO₂ to have a favorable effect in terms of a syngas manufacturing yield and environmental pollution prevention, as compared with Comparative Examples 1 and 2.

In Example 2, as the temperature conditions were changed, H₂ and CO production amounts were best with H₂ being 1128 lbmol/hr and CO being 574 lbmol/hr, and the molar ratio of H₂:CO satisfied about 2:1.

It was confirmed in Example 3 that since the reaction was performed without measuring the flow rate of carbon monoxide converted in the second reactor in real time, the molar ratio of H₂:CO did not satisfy 2:1 and the production amounts of H₂ and CO were somewhat lower than those of Example 1 with H₂ being 1101 lbmol/hr and CO being 478 lbmol/hr, but were higher than those of Comparative Examples 1 and 2.

Since in Example 4, the reaction was performed without performing the regeneration process of the separated catalyst and without recirculation of the catalyst, it was confirmed that the production amount of H₂ and CO were somewhat lower than those of Example 1 with H₂ being 930 lbmol/hr and CO being 459 lbmol/hr as compared with those of Example 1, but were higher than those of Comparative Examples 1 and 2.

Since in Comparative Example 1, the heat treatment process was performed without the catalyst to decrease the simultaneous reaction efficiency of gasification and methane reformation, it was confirmed that the production amounts of H₂ and CO were much lower than those of Example 1 with H₂ being 725 lbmol/hr and CO being 412 lbmol/h.

Since in Comparative Example 2, the reaction was performed without performing the reverse Boudouard reaction by using the manufacturing apparatus of syngas including no second reactor, it was confirmed that the CO production amount was much lower and the CO₂ production amount was much higher than those of Example 1 with H₂ being 925 lbmol/hr, CO being 210 lbmol/hr, and CO₂ being 884 lbmol/hr, and thus, it is not preferred in terms of a manufacturing yield of syngas and environmental pollution.

The method and the apparatus for manufacturing syngas according to the present disclosure may have a significantly improved manufacturing yield of syngas from organic wastes.

The method and the apparatus for manufacturing syngas according to the present disclosure may minimize carbon dioxide formation in a syngas manufacturing process.

The method and the apparatus for manufacturing syngas according to the present disclosure may also induce an effect of regenerating a waste catalyst in a process of converting carbon dioxide into carbon monoxide to improve process efficiency.

Hereinabove, although the present disclosure has been described by specific matters, limited exemplary embodiments, and drawings, they have been provided only for assisting the entire understanding of the present disclosure, and the present disclosure is not limited to the exemplary embodiments, and various modifications and changes may be made by those skilled in the art to which the present disclosure pertains from the description.

Therefore, the spirit of the present invention should not be limited to the above-described exemplary embodiments, and the following claims as well as all modifications equal or equivalent to the claims are intended to fall within the scope and spirit of the invention.

What is claimed is:

1. A method for manufacturing syngas, the method comprising:
(S1) heat-treating organic wastes under a catalyst in a first reactor to produce a first mixed gas;
(S2) separating the catalyst and carbon dioxide (CO₂) from the first mixed gas, and recovering a second mixed gas from which the catalyst and the carbon dioxide (CO₂) have been removed;
(S3) converting the carbon dioxide (CO₂) separated in (S2) into carbon monoxide (CO) by a reverse Boudouard reaction in a second reactor; and
(S4) mixing the second mixed gas recovered in (S2) and the carbon monoxide (CO) converted in (S3) to produce syngas.

2. The method of claim 1, wherein in (S1), when the first mixed gas is produced, a methane reforming reaction in which methane is converted into carbon and hydrogen occurs simultaneously.

3. The method of claim 1, wherein the first mixed gas produced in (S1) comprises hydrogen (H₂), carbon monoxide (CO), and carbon dioxide (CO₂).

4. The method of claim 1, wherein
(S3) further comprises:
(S3-1) introducing the catalyst separated in (S2) to the second reactor and regenerating the catalyst through a reverse Boudouard reaction; and
(S3-2) recirculating and resupplying the regenerated catalyst to (S1).

5. The method of claim 1, wherein in (S1), the catalyst is a composite catalyst in which at least one active metal selected from nickel, iron, or vanadium is supported on a support.

6. The method of claim 1, wherein in (S2), the carbon dioxide (CO₂) is adsorbed on an adsorbent comprising at least one selected from calcium oxide (CaO), calcium hydroxide (Ca(OH)₂), dolomite, limestone, or trona and separated.

7. The method of claim 6, wherein the adsorption of carbon dioxide (CO₂) is performed at a temperature of 500° C. or higher and lower than 800° C. and a pressure of 50 to 200 kPa.

8. The method of claim 1, wherein (S1) is performed at a temperature of 600 to 900° C. and a pressure of 50 to 200 kPa.

9. The method of claim 1, wherein the reverse Boudouard reaction of (S3) is performed at a temperature of 800 to 1000° C. and a pressure of 50 to 200 kPa.

10. The method of claim 1, wherein the syngas produced in (S4) comprises hydrogen (H₂) and carbon monoxide (CO) and a molar ratio between the hydrogen (H₂) and the carbon monoxide (CO) satisfies 1.8 to 2.2.

11. The method of claim 10, wherein the molar ratio between the hydrogen (H₂) and the carbon monoxide (CO) is adjusted by controlling the reverse Boudouard reaction depending on a flow rate of the carbon monoxide (CO) converted in (S3).

12. The method of claim 10, wherein the molar ratio between the hydrogen (H₂) and the carbon monoxide (CO) is adjusted by supplying separate carbon dioxide to (S3) depending on a flow rate of the carbon monoxide (CO) converted in (S3).

13. The method of claim 1, wherein the organic wastes in (S1) are at least one selected from waste plastic, solid wastes, biomass, waste oil, or waste tires.

14. The method of claim 1, further comprising: before (S2), purifying the first mixed gas produced in (S1).

15. An apparatus for manufacturing syngas comprising:
a first reactor where organic wastes are introduced and a methane reforming reaction and a gasification reaction are simultaneously performed under a catalyst to form a product;
a carbon dioxide separation unit where the product is introduced from the first reactor and carbon dioxide is separated;
a second reactor where the carbon dioxide separated from the carbon dioxide separation unit is introduced and a reverse Boudouard reaction is performed; and
a syngas production unit where a product from which the carbon dioxide has been removed in the carbon dioxide separation unit and carbon monoxide converted in the second reactor are mixed to produce syngas.

16. The apparatus of claim 15, wherein the first reactor comprises a fluidized bed reactor.

17. The apparatus of claim 15, wherein the second reactor comprises a fluidized bed reactor.

18. The apparatus of claim 15, further comprising: a purification unit between the first reactor and a carbon dioxide storage unit.

19. The apparatus of claim 15, further comprising:
a cyclone which separates the catalyst from the product of the first reactor between the first reactor and the carbon dioxide separation unit;
a supply line which supplies the catalyst separated from the cyclone to the second reactor; and
a recirculation line which resupplies the regenerated catalyst from the second reactor to the first reactor.

20. A method for manufacturing a liquid hydrocarbon comprising:
supplying syngas to a third reactor; and
performing a Fischer-Tropsch synthesis reaction in the third reactor,
wherein the syngas is the syngas manufactured in claim 1.
